(19) United States
(12) Patent Application Publication
(10) Pub. No.: US 2008/0139906 A1
(43) Pub. Date: Jun. 12, 2008

(54) Device and method for non-invasive optical detection of chemical and physical blood values and body content substances

(76) Inventor: Karlheinz Bussek, Deubach (DE)

Correspondence Address: JENKINS, WILSON, TAYLOR & HUNT, P. A., 3100 TOWER BLVD., Suite 1200, DURHAM, NC, 27707, US

(21) Appl. No.: 11/986,015

(22) Filed: Nov. 19, 2007

(30) Foreign Application Priority Data
Nov. 20, 2006 (DE) 10 2006 054 556.7

(51) Int. Cl. A61B 5/021; A61B 5/00

(52) U.S. Cl. 600/322; 600/485

(57) ABSTRACT

Disclosed are a device and a method for noninvasive determination of body content substances, and to the validation of chemical and physical characteristics of blood and other body fluids. In particular, a noninvasive determination of the blood pressure is disclosed, in connection with other blood values of arterial blood, using optical methods at a patient interface, including the first device in the form of a optical transmitter and a second device in the form of an optical receiver, wherein the optical transmitter and the optical receiver are synchronized with each other by means of a control unit.

### CROSS-REFERENCE TO RELATED APPLICATIONS

This application claims the benefit of German Patent Application No. 10 2006 054 556.7, filed Nov. 20, 2006, the entire disclosure of which is herein incorporated by reference.

### FIELD OF THE INVENTION

The present invention generally relates to a noninvasive determination of body content substances and to a validation of chemical and physical characteristic values of blood and other body fluids. In particular, the invention relates to a noninvasive determination of blood pressure in conjunction with other blood values of arterial blood, using optical measurement methods.

### BACKGROUND OF THE INVENTION

The determination of chemical content substances and the measurement of the blood pressure through various methods are well known, and the various methods are described in the state of the art. Blood pressure measurement devices are known in particular for determining the arterial blood pressure. The heart is the driving force of the blood pressure. During the expulsion phase of the blood (systole), the expelled blood is ejected and stored in the aorta, and delivered to the subsequent vessel system in the pursuant detention phase (diastole). The pressure or the arterial blood pressure thus created is determined and influenced by various factors. These are substantially the volume of the heart and the force of the beat, thus the power of the heart, the elasticity of the arteries, and the peripheral flow resistance of the blood vessels, and the volume of the blood. Current measurement methods can be divided into two groups by principle:
a) continuous method; and
b) discontinuous method.

The discontinuous methods typically are methods, which date back to Riva-Rocci, and which detect the pressure or the pressure change and the associated Korotkow-sounds by means of a membrane and a suitable measurement sleeve, which can either be determined by means of a stethoscope or by means of alternative methods. Thus, the different sounds and the change of these sounds are associated with the systolic and diastolic blood pressure. There are diverse refinements of this method, which in principle are all based on the same base principle of pressure measurement and measurement of pressure variations.

The disadvantage of these methods is the sensation of the patient. Inflating the sleeve is uncomfortable for the patient and can even become painful at higher sleeve pressures. Additionally, the measurement cannot be repeated permanently, since damages to the artery can occur, so that the continuous detection of the blood pressure is not possible, which is disadvantageous. Mandatory measurement pauses have to be taken. Additionally, the method of inflating and deflating air from the sleeve is time consuming and involved. A desired continuous detection of measurement values and data with reference to blood values is not possible with the known methods. However, it is desirable to detect the blood pressure and other vital values and body content substances simultaneously and additionally possibly permanently and continuously.

In the continuous methods, like e.g. the volume compensation method and the arterial applantation tonometry, other disadvantages are associated with the measurement method. In case of the volume compensation method, the patient suffers pain, in particular when measuring at the finger. Additionally, phenomena occur during restricted peripheral bleeding, like e.g. in case of shock or shock type conditions, which distort the measurement of the blood pressure and cause an imprecise measurement. Partially, especially with such patients, in particular in critical phases, a measurement, which is as precise and simple as possible, and a fast detection of as many measurement data as possible, like e.g. blood pressure and vital values, is desirable and necessary. In arterial applantation tonometry, however, motion artifacts are an obstacle with reference to the absolute measurement values and the measurement precision resulting there from. The measurement results are rather unsatisfactory, if the patient is not held completely still. A frequent adjustment and the recalibration of the measurement device is already necessary in case of minor movements of the sensors at the patient interface.

All known methods, however, besides the problem of the artifacts, also have the common problem that the required values can only be measured in an isolated manner. Timewise correlated information with respect to chemical concentration values of the blood are missing, in particular values from the noninvasive determination of vital parameters and biophysical variables, like blood sugar and other elementary components of the blood, or of body fluids to be analyzed. Independent from the blood pressure it can also be necessary to determine only the vital values, for which the known methods are not suited, since they are configured only for determining blood pressure values.

Noninvasive methods for determining vital values and constant content substances of fluids are also known in the state of the art. In this context, the measurement of the oxygen saturation level has to be emphasized in particular.

Thus, light in the visible and in the near infrared range of the electromagnetic spectrum is used for measurements of the oxygen saturation levels in the blood of the patient. For this purpose, spectrometric instruments in conjunction with suitable-typically plural sensor surfaces are used, by means of which the oxygen saturation of blood is estimated or calculated in an approximation.

Different methods for pulse oximetry are known, in order to determine the oxygen saturation value of arterial blood as precisely as possible.

Furthermore, it is known that the arterial oxygen saturation can be determined through isolation of the change in the detected light intensities during a heart cycle, and the attempt to minimize or even eliminate the absorption effects of non-arterial blood tissue of the patient. Though, this type of oximetry measurement attempts to eliminate a plurality of artifacts like influences of bones, skin and muscles and so forth, the disadvantage is that the signal reception circuits and the analysis circuits have to be provided very robust, since the usable and processible part of the measurement signal is the relatively small change of the determined and detected intensities, and it is small relative to the entire detected intensity. Additionally, the measurement results are influenced by pulsating signal contributions from many proximal tissue layers, so that the determined measurement value of the oxygen saturation is rather imprecise and can be distorted.

Further methods of spectral analysis and differential optical determination of the oxygen content of human blood are known, which, however, all comprise the disadvantages of artifacts, and which do not provide any contribution for detecting the blood pressure or with respect to other chemical content substances of the blood. Another disadvantage is that the said measurement methods are not suitable for simultaneously detecting the blood pressure of the patient, or of the person to be examined. For this purpose, a parallel and additionally time consuming method would be required, in order to measure the blood pressure. Other vital values also cannot be determined either with the known measurement devices and the methods described herein.

All known methods and devices have the disadvantages of measurement imprecision in common, and the influence of artifacts, and in particular the disadvantage, that it is not possible to determine different content substances with an identical method. Besides that, some methods of blood pressure measurement are painful and inconvenient.

In medicine and during patient supervision, the permanent control of the blood pressure and the repeated determination of blood values and body content substances are typically very important.

The present method also attempts to circumvent additional disadvantages of established methods, besides the above mentioned disadvantages. For example, the elimination of the inconveniences for the patients compared to the invasive subtraction of body fluids like blood, and the reduction of the risk of infection, which constitutes a latent risk in the established invasive methods, is a decisive advantage and furthermore offers the additional potential to reduce the time required for a measurement, and thus the expenses associated therewith.

Furthermore, often not only particular values like the blood pressure, or the sodium content of the blood need to be determined, but additionally also a number of measurement values is required, so that they have to be determined with the traditional established methods, one after other, or with different measurement devices simultaneously. The changing of the measurement devices creates additional disadvantages for the reference measurement values.

### SUMMARY OF THE INVENTION

Therefore, it is an object of the present invention to provide a method and a device for improved noninvasive measurement of body content substances and blood measurement values and of blood pressure. This object is accomplished on the device side by the features of claim 1 and on the method side by the features of claim 21.

The invention is based on the basic finding that the electrochemical processes in the human heart and body and therefore the actual chemical composition of the blood of a person to be examined, or of a patient are in direct relationship and thus in a measurable dependency from the arterial blood pressure of the patient, or they change accordingly therewith. In particular, the local concentration of chemical elements of the blood depends on the actually present arterial blood pressure. For example, the sodium or potassium content changes in correlation with the blood pressure. This relationship between the concentration of content materials and the arterial blood pressure is a matter of principle.

Furthermore, the invention is based on the physical principles and laws of the propagation of electromagnetic waves, in particular on the principles of the occurring interactions of the light waves in the visible, UV- and infrared range, when passing through liquid bodies. In this context, the Lambert-Beer-law should be mentioned, which, on the one hand, describes the relationship of total absorption of a medium, depending on the concentration of the substances included, and, on the other hand, addresses the phenomenon, that there is no mathematical context between the emitted and absorbed light intensity of a light wave, which passes through such a substance.

The method and the device according to specific embodiments of the present invention are based on the principle of spectroscopic sensing, using suitable narrow band tunable light sources, or generally a narrow band tunable actor system.

An embodiment of the present invention combines an optical tunable actor system, in particular an optically tunable light source, comprising an optically tunable sensor system, in particular an optically tunable receiver. According to one aspect of the invention, the entire visible and non-visible frequency range of the optical spectrum is used in the optically tunable actor system for determining the desired measurement data in the optically tunable actor system, in order to obtain the desired measurement values in the optical sensor system through an optical method. In one embodiment, the actor system and sensor system, or the transmitter and the receiver are synchronized with each other through a control unit. Besides a single actor system or sensor system, also several transmitters and receivers, and a transmitter with several receivers and several transmitters can be combined with one receiver in the respective actor system or sensor system, and can be synchronized relative to each other in a suitable manner according to the measurement task.

Thus, the blood of the patient to be analyzed is irradiated at the patient interface (e.g. finger, suitable body section or tissue) for example with monochromatic electromagnetic radiation by means of the optically tunable transmitter of the actor system. The optically tunable transmitter can thus be a narrow band light source, which can be tuned in the required spectral range, and the receiver can be an optical spectrum analyzer, which can be tuned in the respective spectral range. The processing of the spectral lines of the optical actor system and sensor system yields the desired measurement values of the analyzed blood components or of the analyzed body fluids, using suitable processing analytics, wherein said measurement values result from the validation of the detected reception signals in the receiver for the respective patient. In order to be free from artifacts, additionally a calibration process of the measurement amplitudes can be performed. Through the synchronization and the knowledge of the normal amplitude with the reference to the measured light intensity, a numeric multiplier can be found which results from the relationship of the measured amplitude to the normal amplitude, in order to perform a calibration process therewith. Movements and other movement dependent artifacts are eliminated as a consequence of the synchronization of the transmit/receive distance.

Alternatively, besides the already mentioned body fluids, or the blood also suitable tissue components can be used, in order to apply the method according to the invention, and in order to determine the desired vital values, body content substances, and the desired measurement values.

A device, which combines the method according to an embodiment of the present invention thus comprises an optical transmitter device and an optical receiver device and of a processing device, for example an electronic processing device, validating the relationships of the emitting spectral lines of the transmitter and of the detected spectral lines in the receiver device of the receiver, according to suitable mathematical methods, and putting them out as numerical values. A possible output form is a digital output of the measurement values, calculated according to the calibration method. Alternatively, also interfaces can be provided, which compute the determined signals from the receiver into desired output formats, and which put them out e.g. in the form of graphic tables or graphics.

Further embodiments can be derived from the additional dependent claims.

Subsequently, preferred embodiments of the invention are described in more detail with reference to the figures of the drawing.

### BRIEF DESCRIPTION OF THE DRAWINGS

FIG. 1 shows a schematic block diagram illustrating the basic method and schematically illustrating a device for determining body content substances and blood values;

FIG. 2 shows a schematic block diagram similar to FIG. 1, however, with two optically tunable transmitters and two optically tunable receivers and a processing device;

FIG. 3 also schematically shows a block diagram similar to FIGS. 1 and 2, however with an optical prism;

FIG. 4 shows the relationship of a medication with the detected spectral line after the absorption;

FIG. 5 shows the principle spectral distribution of different materials with reference to the wavelength and their characteristic absorption bands;

FIG. 6 shows the spectrum of the light with the tunable wavelength range, which is necessary for the present method;

FIG. 7 shows the features of an ideal tunable light source with a spectral band in an exemplary manner; and

FIG. 8 schematically shows the block diagram similar to FIG. 1 with a finger as an example for a patient interface.

FIG. 1 shows a transmitter device 1, comprising a suitable tunable light source in the form of an optically tunable transmitter 3. The transmitter 3 comprises a first device 10, e.g. a monochromator for generating monochromatic light in the form of electromagnetic radiation 22, which is generated in the optically tunable transmitter 3. The transmitter 3 is oriented relative to a patient interface 23, so that the monochromatic electromagnetic radiation exiting from the tunable transmitter penetrates into the patient interface 23 and passes through it, like in the present example, or it is scattered thereon. Thereby, an interaction occurs, which is desired and significant for each substance, wherein said interaction occurs with the content substances to be measured, depending on their presence and concentration. The radiation 32 exiting from the patient interface, wherein said radiation is transmitting in the present case, is detected in a receiver device 2 by a receiver 4, which is optically tunable. The optically tunable transmitter 3 and the optically tunable transmitter 4 are synchronized relative to each other through a control unit 5, which controls and/or regulates the emission frequencies of the transmitters 3 and 4. A device is designated with the reference numeral 11, which tunes the receiver 4 continuously is variable. In the present case, e.g. an optical grid 12 was used.

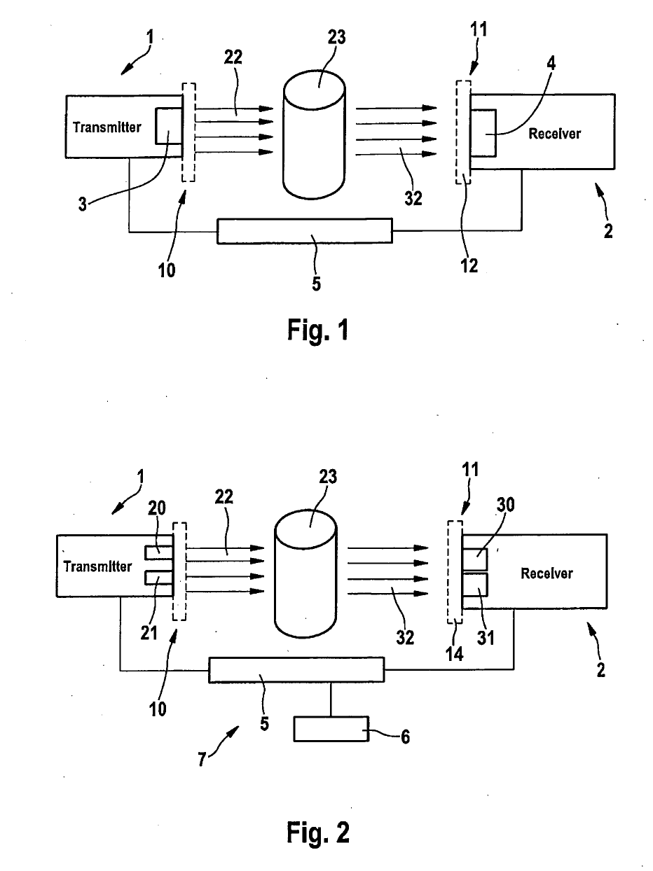

FIG. 2 shows a second embodiment with a setup similar to FIG. 1. In this embodiment, however, the transmitter 3 has two separate light sources, which can operate in parallel as optically tunable transmitters 20, 21. A comparable arrangement is shown on the receiver side. Here, two optically tunable receivers 30, 31 are disposed separate from each other, but close to each other.

The method can also be performed by only one of the two receivers, depending on the measurement task. The illustrated receivers 30, 31 can e.g. be optically tunable narrow band catalytic spectrum analyzers. The control unit 5 synchronizing the transmitters 20, 21 with the receivers 30, 31, either in pairs, or selectively synchronizes the transmitter 20, 21, e.g. with a receiver 30, thus offers the opportunity to also process signals, that are absolutely differential to each other, and to compare the received signals of two simultaneously transmitted light frequencies of the transmitter 20, 21 with each other. Using the Doppler Effect, e.g. flowing substances in the patient interface 23 can be determined by means of a differential method through the processing unit 6.

The processing unit 6 is used for calibration, analysis and validation of the determined measurement values, based on mathematical methods. An electronic narrow band filter 14 takes over the task of the device 11 from FIG. 1. It is conceivable to house the control unit 5 and the processing unit 6 in a joint control and processing device 7.

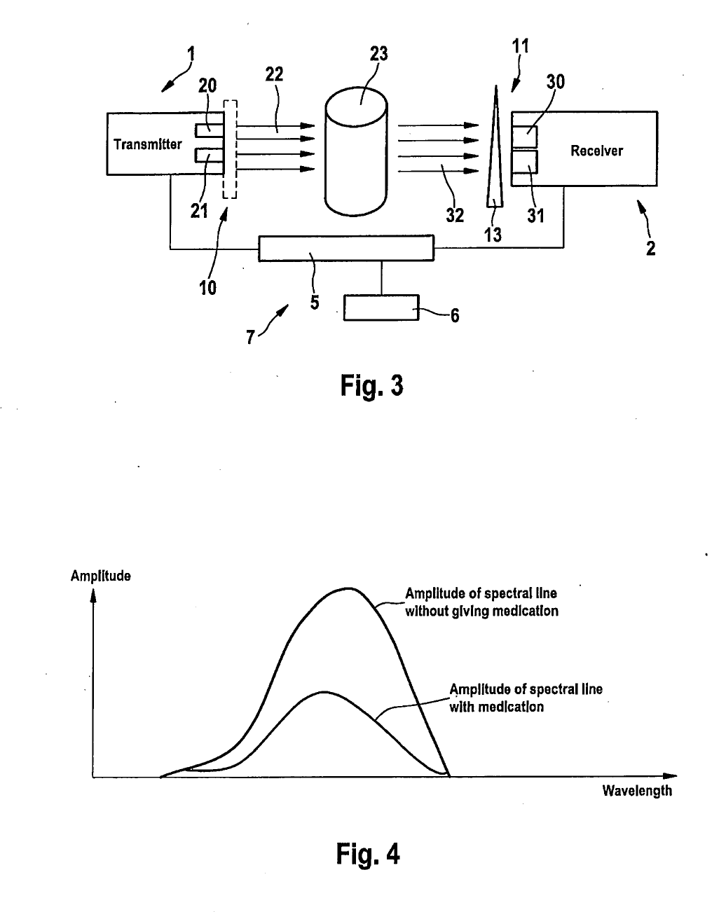

FIG. 3 shows a third embodiment similar to the one of FIG. 2. Here, an optical prism 13 is illustrated in connection with the receivers 30, 31, which constitute an alternative possibility besides a refractive grid of the device 11 from FIG. 2.

The graphics shown in FIG. 4 constitute the context between a detected light frequency of a certain intensity and the amplitude of the spectral line (upper curve), when no medication is present. In this case, the medication is missing, so that therefore no absorption effects and interactions occur in the patient interface 23 with certain content substances, which respond to the medication (e.g. potassium concentration) in a significant wavelength range. The preferred method, however, detects a lower signal by means of the receiver, synchronously tuned to the transmitter, after delivering medication after passing through the patient interface 23 (e.g. the finger of a person to be tested), within the significant wavelength range (lower curve).

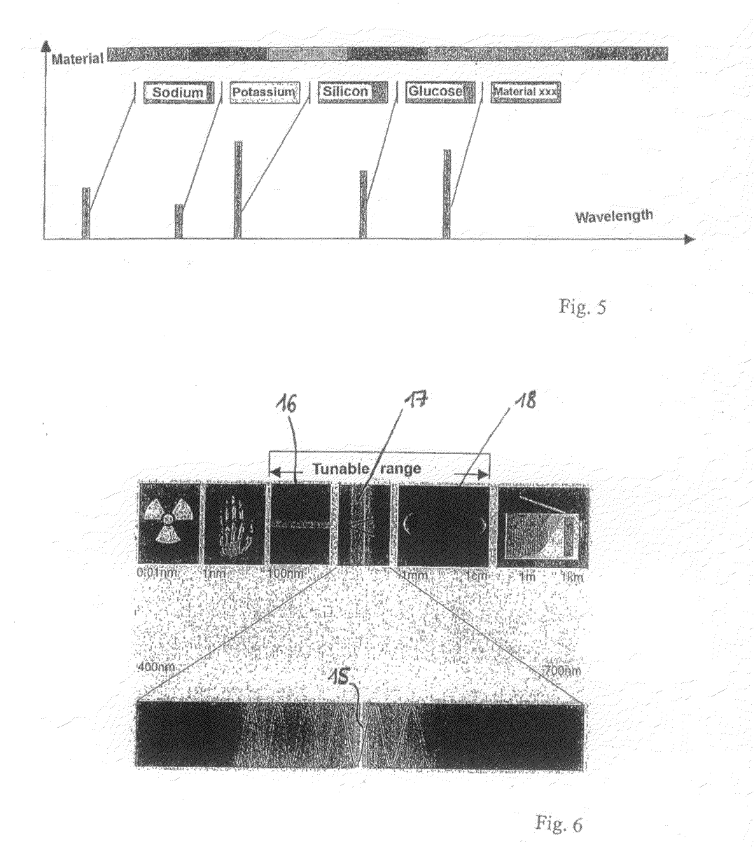

Chemical substances and elements show a spectral distribution, which is typical for these substances and elements. In FIG. 5, the principal spectral distribution is illustrated, dependent on the wavelength, in an exemplary manner for the substances sodium, potassium, silicon, glucose, and another exemplary substance. The height of the amplitude is thus variable and depends on diverse factors, like e.g. the irradiated light power, and also depends on the concentration of the materials concerned.

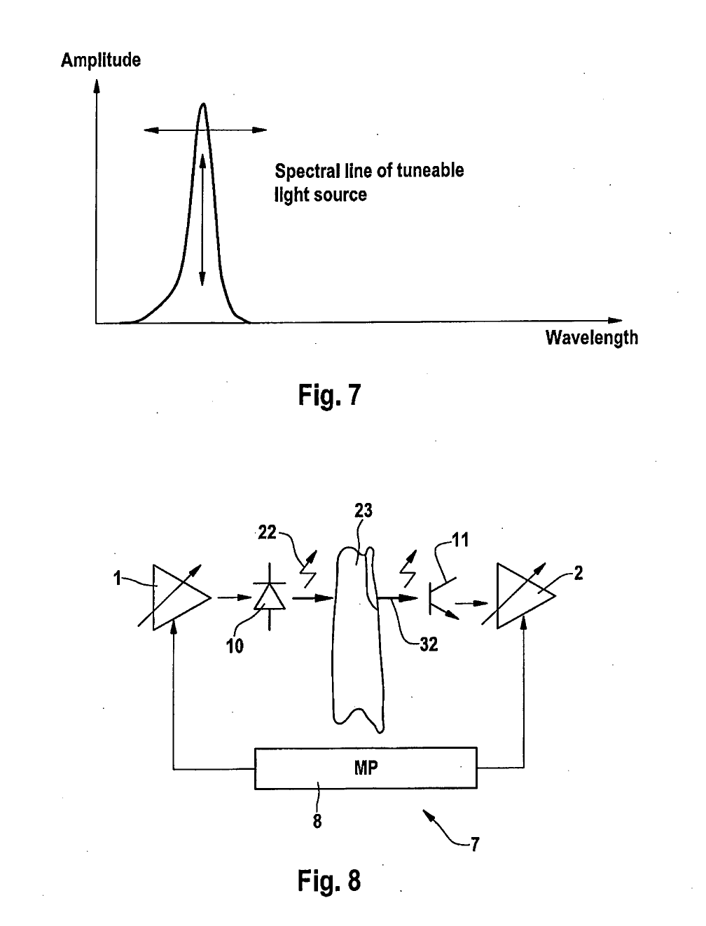

In an exemplary manner, the light spectrum is shown in FIG. 6, and plotted over the wavelength. The spectral range that is used can be divided based on FIG. 6 into the three following ranges, over which the optical transmitters 3, 20, 21, and the optical receivers 4, 30, 31, can be tuned: UV range 16, visible range 17, and IR range 18. Typical values are disposed approximately between 100 nm and 1 cm. The term of the tunable light source is described in more detail in FIG. 7. Here, the amplitude of a discretional spectral line is plotted over the wavelength. The horizontal and vertical arrows indicate the ranges of the tunable factors, like amplitude and frequency, or wavelength.

FIG. 8 shows a circuit layout similar to the one shown in FIG. 1. A microprocessor 8 can be used for this task of handling the occurring high data volumes, and the high computation speed thereby required and simultaneously for the fast adaptation during the synchronization of the transmitter-receiver-distance.

What is claimed is:

1. A device for noninvasive optical detection of chemical and physical blood values and body content substances at a patient interface comprising:
at least one transmitter device in the form of an optically tunable transmitter and
at least one associated receiver device in the form of an optically tunable receiver,
wherein the at least one optically tunable transmitter and the at least one associated optically tunable receiver are synchronized relative to each other by means of an associated control unit.

2. The device according to claim 1, wherein a blood pressure can be determined by means of data detected at the optically tunable receiver.

3. The device according to claim 1, wherein the control unit and a processing unit are combined into a control and processing unit.

4. The device according to claim 1, wherein the optical transmitter is continuously variably tunable by means of a first device over a frequency range.

5. The device according to claim 1, wherein the optical receiver is continuously variably tunable by means of a second device over a frequency range.

6. The device according to claim 4, wherein the optical transmitter is continuously variably tunable by means of the first device from the UV frequency range over the visible frequency range to the infrared frequency range.

7. The device according to claim 5, wherein the optical receiver is continuously variably tunable by means of the second device from the UV frequency range over the visible frequency range to the infrared frequency range.

8. The device according to claim 1, wherein the optical receiver is continuously variably tunable by means of an optical grid over a frequency range.

9. The device according to claim 1, wherein the optical receiver is continuously variably tunable by means of an optical prism over a frequency range.

10. The device according to claim 1, wherein the optical receiver is continuously variably tunable by means of electronic narrow band filters over a frequency range.

11. The device according to claim 1, wherein the optical transmitter emits a narrow band, almost monochromatic light.

12. The device according to claim 1, wherein the optical transmitter emits coherent light.

13. The device according to claim 1, wherein the optical transmitter emits polarized light in addition to non-polarized light.

14. The device according to claim 1, wherein the transmitter comprises one or several optical transmission units, or separate optically tunable transmitters.

15. The device according to claim 1, wherein the receiver comprises several optical receiver units, or separate optically tunable receivers.

16. The device according to claim 14, wherein the optical transmitters are synchronized respectively to at least one of the two optical receivers by means of the control unit.

17. The device according to claim 16, wherein the control unit determines the chemical and physical values of the body content substances and the blood pressure from the detected differential signals of at least one optically tunable receiver from different signals of the optically tunable transmitters by means of a differential analysis method.

18. The device according to claim 17, wherein the control and processing unit determines additional values with respect to the flow conditions within the body fluids by means of the Doppler effect.

19. The device according to claim 1, wherein the synchronization of the optically tunable transmitter(s) and of the optically tunable receiver(s) and the validation of the determined signals is performed by means of a control unit and a microprocessor.

20. The device according to claim 1, wherein the receiver is a spectrum analyzer with an integrated processing device.

21. A method for optical detection of chemical and physical blood values and body content substances at a patient interface by means of at least one optically tunable transmitter and at least one associated optically tunable receiver, using a control unit for synchronizing the at least one transmitter and the at least one associated receiver with each other, comprising the following steps:
a) imparting a narrow band electromagnetic radiation into the patient interface by means of the at least one optically tunable transmitter;
b) validating the signals detected after passing the patient interface in the at least one associated optically tunable receiver; and
c) calibrating the determined measurement data by means of a calibration process.

22. The method according to claim 21, wherein the correlated arterial blood pressure is validated and determined by means of the control unit from the signals detected in the receiver unit and from the concentration values of body content substances resulting there from.

23. The method according to claim 21, wherein the method is performed by means of at least one synchronized and optically tunable transmitter and at least one associated synchronized and optically tunable receiver, using a device comprising the features according to one of the claims 1 through 20.
